(19) United States
(12) Patent Application Publication
(10) Pub. No.: US 2026/0195890 A1
(43) Pub. Date: Jul. 9, 2026

(54) HIERARCHICAL SEGMENTATION OF SURGICAL SCENES

(71) Applicant: DIGITAL SURGERY LIMITED, London (GB)

(72) Inventors: Imanol LUENGO MUNTION, London (GB); Pritesh MEHTA, London (GB); David P. OWEN, London (GB); Danail V. STOYANOV, London (GB); Maria GRAMMATIKOPOULOU, London (GB)

(21) Appl. No.: 19/129,543

(22) PCT Filed: Nov. 14, 2023
(86) PCT No.: PCT/EP2023/081794

§ 371 Date: May 13, 2025

(30) Foreign Application Priority Data
Nov. 14, 2022 (GR) 20220100936
Jun. 13, 2023 (GR) 20230100471

(51) Int. Cl. G06T 7/00; A61B 34/20; G06T 7/11; G06T 7/174; G06V 10/26; G06V 10/764; G06V 10/776; G06V 10/82; G06V 20/40; G06V 20/70

(52) U.S. Cl. CPC G06T 7/0012; A61B 34/20; G06T 7/11; G06T 7/174; G06V 10/764; G06V 20/41; A61B 2034/2055; A61B 2034/2065; G06T 2207/10016; G06T 2207/10068; G06T 2207/20016; G06T 2207/20076; G06T 2207/20081; G06T 2207/20084; G06T 2207/30101; G06V 10/26; G06V 10/776; G06V 10/82; G06V 20/70; G06V 2201/034

(57) ABSTRACT

A computer-implemented method for performing a hierarchical segmentation of video frames in surgical videos includes obtaining an image of an anatomical structure, where the image includes a plurality of image pixels, generating a multi-label probability map for each node of a pre-defined hierarchy of segmentation classes, processing the plurality of image pixels to generate a leaf-level segmentation map, and processing each leaf-level segmentation and updating a class label for each leaf-level segmentation to a higher parent class until a prediction confidence threshold is achieved.

### BACKGROUND

The present disclosure relates in general to computing technology and relates more particularly to computing technology for the hierarchical segmentation of video frames in surgical videos.

Computer-assisted systems, particularly computer-assisted surgery systems (CASs), rely on video data digitally captured during surgery. Such video data can be stored and/or streamed. In some cases, video data can be used to augment a person's physical sensing, perception, and reaction capabilities. For example, such systems can effectively provide the information corresponding to an expanded field of vision, both temporal and spatial, that enables a person to adjust current and future actions based on the part of an environment not included in his or her physical field of view. Alternatively, or in addition, the video data can be stored and/or transmitted for several purposes, such as archival, training, post-surgery analysis, and/or patient consultation.

Segmentation of surgical scenes may provide valuable information for real-time guidance and post-operative analysis of robotic-assisted laparoscopy. Unfortunately, however, segmentation of surgical video frames is challenging due to ambiguities caused by similar appearances of anatomical structures; occlusion by blood, visceral fat, and/or smoke, and reduced anatomical reference due to camera pose. This leads to missed detections or incorrect predictions of anatomical class.

### SUMMARY

According to an aspect, a computer-implemented method for performing a hierarchical segmentation of video frames in surgical videos is provided. The method includes obtaining an image of an anatomical structure, where the image includes a plurality of image pixels, generating a multi-label probability map for each node of a pre-defined hierarchy of segmentation classes, processing the plurality of image pixels to generate a leaf-level segmentation map, and processing each leaf-level segmentation to update the class label for each leaf-level segmentation to a higher parent class until a pre-determined prediction confidence threshold is achieved.

According to another aspect, a system includes a data store including video data associated with a surgical procedure and a machine learning training system for training a hierarchical model to perform hierarchical segmentation of video frames in surgical videos. The system is configured to obtain an image of an anatomical structure from the video data, wherein the image includes a plurality of image pixels, generate a multi-label probability map for each node of a pre-defined hierarchy of segmentation classes, process the plurality of image pixels to generate a leaf-level segmentation map, and process each leaf-level segmentation to update the class label for each leaf-level segmentation to a higher parent class until a pre-determined prediction confidence threshold is achieved.

According to an aspect, a computer program product is provided and includes a memory device having computer executable instructions stored thereon, which when executed by one or more processors cause the one or more processors to perform a plurality of operations for performing a hierarchical segmentation of video frames in surgical videos. The plurality of operations include obtaining an image of an anatomical structure, where the image includes a plurality of image pixels, generating a multi-label probability map for two or more nodes of a pre-defined hierarchy of segmentation classes, processing the plurality of image pixels to generate a leaf-level segmentation map, and updating a class label for at least one leaf-level segmentation to a higher parent class until a pre-determined prediction confidence threshold is achieved.

The above features and advantages, and other features and advantages, of the disclosure are readily apparent from the following detailed description when taken in connection with the accompanying drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the aspects of the disclosure are apparent from the following detailed description taken in conjunction with the accompanying drawings in which:

FIG. 1 depicts a computer-assisted surgery (CAS) system according to one or more aspects;

FIG. 2 depicts a surgical procedure system according to one or more aspects;

FIG. 3 depicts a system for analyzing video and data according to one or more aspects;

FIG. 4A depicts a visual flow diagram showing a hierarchical model inference for a laparoscopic frame, according to one or more aspects;

FIG. 4B depicts a visual flow diagram showing mixing for a cystic artery using a trained hierarchical model, according to one or more aspects;

FIG. 5 depicts a hierarchy chart which may be used for hierarchical training and inference, according to one or more aspects;

FIG. 6 depicts a visual comparison of a categorical cross-entropy (CCE) baseline and a hierarchical training and inference, according to one or more aspects;

FIG. 7 depicts a visual comparison of the CCE baseline and the hierarchical training and inference across a temporal sequence of frames, according to one or more aspects;

FIG. 8 depicts a flowchart of a method of performing a hierarchical segmentation of video frames in surgical videos, according to one or more aspects; and

FIG. 9 depicts a block diagram of a computer system, according to one or more aspects.

The diagrams depicted herein are illustrative. There can be many variations to the diagrams and/or the operations described herein without departing from the spirit of the described aspects. For instance, the actions can be performed in a differing order, or actions can be added, deleted, or modified. Also, the term “coupled” and variations thereof describe having a communications path between two elements and do not imply a direct connection between the elements with no intervening elements/connections between them. All of these variations are considered a part of the specification.

### DETAILED DESCRIPTION

Exemplary aspects of the technical solutions described herein include systems and methods for the hierarchical segmentation of video frames in surgical videos.

In order to improve segmentation in analyzing surgical videos, aspects of technical solutions are described herein and may use a segmentation hierarchy and an associated hierarchical inference scheme which allows for grouped anatomic structures to be predicted when fine-grained classes cannot be reliably distinguished. This disclosure provides unique and novel technical solutions which are rooted in computing technology and which provides improvement over current segmentation abilities to achieve better results than current segmentation art. In this disclosure, a multi-label segmentation loss informed by a hierarchy of anatomic classes is formulated, and a network is trained using this hierarchy. Subsequently, a leaf-to-root inference scheme (“Hiera-Mix”) may be used to determine a trade-off between label confidence and granularity in a given scene. This method may be applied to any segmentation model and may be evaluated using a large dataset, such as a laparoscopic cholecystectomy dataset with 65,000 labelled frames, as one example.

Technical solutions are described herein to address such technical challenges. Particularly, technical solutions herein may facilitate improved segmentation and detection accuracy of “critical structures” (e.g., cystic artery and cystic duct) when evaluated across hierarchy paths. This may correspond to visibly improved segmentation outputs, with fewer interclass confusions. For other anatomic classes, which benefit less from the hierarchy, segmentation and detection are unimpaired. Moreover, embodiments described herein provide a hierarchical approach that improves surgical scene segmentation in frames with ambiguous anatomy. This may be accomplished by more suitably reflecting a model's parsing of the scene and may be beneficial in applications of surgical scene segmentation, including advancements in computer-assisted intra-operative guidance.

Laparoscopic cholecystectomy is a minimally invasive surgical procedure which that can be used to remove a gallbladder. This procedure involves the dissection of a “critical area” to expose the “critical structures” that are keeping the gallbladder attached (e.g., cystic artery and cystic duct) to the body, and clipping and dividing these critical structures once they are exposed. However adverse outcomes, including death, can occur during this procedure. In a small number of cases, major bile duct injury may occur due to an accidental division of the common bile duct as opposed to the cystic duct. Accordingly, official guidance encourages surgeons to establish a “Critical View of Safety” (CVS) before clipping and dividing the critical structures. Once CVS is achieved, both the cystic artery and cystic duct are separated and clearly distinguishable so that the cystic artery and the cystic duct can be seen as the only two structures entering the gallbladder and thus, can be easily traced as they enter the gallbladder.

Robotic-Assisted Surgery (RAS), such as robotic laparoscopic surgery, has enabled surgeons to perform minimally invasive procedures with greater precision, thereby resulting in less pain, scarring, reduced blood loss, and faster recovery. A key component of RAS which allows for increased surgical precision is visual feedback via integrated imaging and display technology. This technology is typically capable of providing the surgeon with a high resolution, magnified view of the internal anatomy of interest and the surgical tools being used. Furthermore, solutions have been developed to allow post-operative analysis of recorded surgical video. However, interpretation of surgical video can be challenging due to several reasons, including but not limited to, occlusion caused by blood, visceral fat, inflammation, smoke (e.g., from electrocautery), as well as other anatomical structures that are not the target structures of interest, reduced anatomical reference due to magnification/camera angle, and specularity. Deep learning-based semantic segmentation of anatomical structures in surgical video frames provides potential to enhance surgical safety and workflow.

According to embodiments disclosed herein, Hierarchical Semantic Segmentation (HSS) for segmenting anatomy in surgical video frames may be used for any segmentation problem where classes can be arranged in a hierarchy. HSS may improve segmentation performance in computer vision datasets, such as street-scene parsing and human body parsing. Performance improvements may also be achieved by imposing a hierarchy on anatomical classes. A hierarchical inference method (“Hiera-Mix”) can predict grouped confusable structures until such point that they can be confidently distinguished from one another. The embodiments disclosed herein facilitate the hierarchical approach in improving cross-dissection segmentation of the critical structures: the cystic artery and cystic duct, as well as the undissected fat that covers the cystic artery and cystic duct and the common bile duct below the cystic artery and cystic duct.

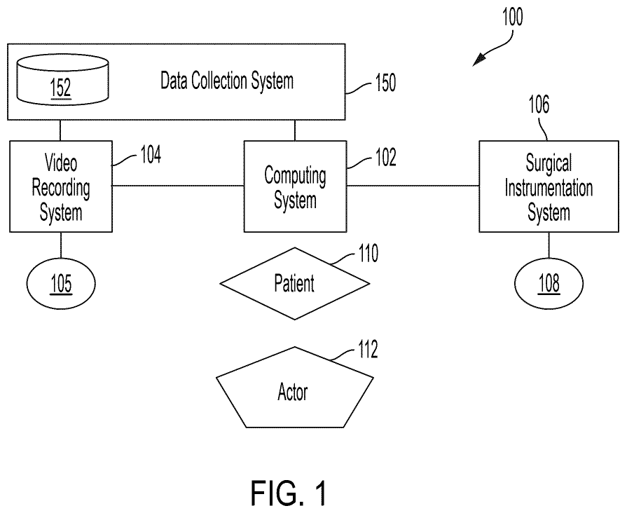

Turning now to FIG. 1, an example computer-assisted system (CAS) system 100 is generally shown in accordance with one or more aspects. The CAS system 100 includes at least a computing system 102, a video recording system 104, and a surgical instrumentation system 106. As illustrated in FIG. 1, an actor 112 can be medical personnel that uses the CAS system 100 to perform a surgical procedure on a patient 110. Actor 112 may be any medical personnel such as a surgeon, assistant, nurse, administrator, or any other actor that interacts with the CAS system 100 in a surgical environment. The surgical procedure can be any type of surgery, such as but not limited to cataract surgery, laparoscopic cholecystectomy, endoscopic endonasal transsphenoidal approach (eTSA) to resection of pituitary adenomas, or any other surgical procedure. In other examples, actor 112 can be a technician, an administrator, an engineer, or any other such personnel that interacts with the CAS system 100. For example, actor 112 can record data from the CAS system 100, configure/update one or more attributes of the CAS system 100, review past performance of the CAS system 100, repair the CAS system 100, and/or the like including combinations and/or multiples thereof.

A surgical procedure can include multiple phases, and each phase can include one or more surgical actions. A “surgical action” can include an incision, a compression, a stapling, a clipping, a suturing, a cauterization, a sealing, or any other such actions performed to complete a phase in the surgical procedure. A “phase” represents a surgical event that is composed of a series of steps (e.g., closure). A “step” refers to the completion of a named surgical objective (e.g., hemostasis). During each step, certain surgical instruments 108 (e.g., forceps) are used to achieve a specific objective by performing one or more surgical actions. In addition, a particular anatomical structure of the patient may be the target of the surgical action(s).

The video recording system 104 includes one or more cameras 105, such as operating room cameras, endoscopic cameras, laparoscopic cameras, and/or the like including combinations and/or multiples thereof. The cameras 105 capture video data of the surgical procedure being performed. The video recording system 104 includes one or more video capture devices that can include cameras 105 placed in the surgical room to capture events surrounding (i.e., outside) the patient being operated upon. The video recording system 104 further includes cameras 105 that are passed inside (e.g., endoscopic cameras) the patient 110 to capture endoscopic data. The endoscopic data provides video and images of the surgical procedure.

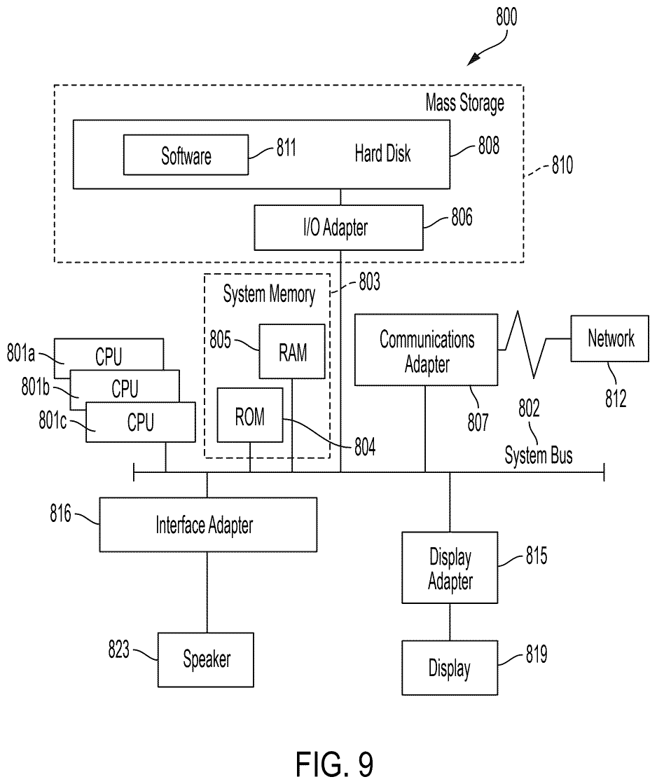

Computing system 102 includes one or more memory devices, one or more processors and a user interface device, among other components. All or a portion of the computing system 102 shown in FIG. 1 can be implemented, for example, by all or a portion of computer system 800 of FIG. 9. Computing system 102 can execute one or more computer-executable instructions. The execution of the instructions facilitates the computing system 102 to perform one or more methods, including those described herein. Computing system 102 can communicate with other computing systems via a wired and/or a wireless network. In one or more examples, the computing system 102 includes one or more trained machine learning models that can detect and/or predict features of/from the surgical procedure that is being performed or has been performed earlier. Features can include structures, such as anatomical structures, surgical instruments 108 in the captured video of the surgical procedure. Features can further include events, such as phases and/or actions in the surgical procedure. Features that are detected can further include the actor 112 and/or patient 110. Based on the detection, the computing system 102, in one or more examples, can provide recommendations for subsequent actions to be taken by the actor 112. Alternatively, or in addition, the computing system 102 can provide one or more reports based on the detections. The detections by the machine learning models can be performed in an autonomous or semi-autonomous manner.

Machine learning models can include artificial neural networks, such as deep neural networks, convolutional neural networks, recurrent neural networks, vision transformers, encoders, decoders, or any other type of machine learning model. Machine learning models can be trained in a supervised, unsupervised, or hybrid manner. The machine learning models can be trained to perform detection and/or prediction using one or more types of data acquired by the CAS system 100. For example, machine learning models can use the video data captured via the video recording system 104. Alternatively, or in addition, the machine learning models use the surgical instrumentation data from the surgical instrumentation system 106. In yet other examples, the machine learning models use a combination of video data and surgical instrumentation data.

Additionally, in some examples, the machine learning models can also use audio data captured during the surgical procedure. The audio data can include sounds emitted by the surgical instrumentation system 106 while activating one or more surgical instruments 108. Alternatively, or in addition, the audio data can include voice commands, snippets, or dialog from one or more actors 112. The audio data can further include sounds made by the surgical instruments 108 during their use.

In one or more examples, the machine learning models can detect surgical actions, surgical phases, anatomical structures, surgical instruments, and various other features from the data associated with a surgical procedure. The detection can be performed in real-time in some examples. Alternatively, or in addition, computing system 102 analyzes the surgical data, i.e., the various types of data captured during the surgical procedure, in an offline manner (e.g., post-surgery). In one or more examples, the machine learning models detect surgical phases based on detecting some of the features, such as the anatomical structure, surgical instruments, and/or the like including combinations and/or multiples thereof.

A data collection system 150 can be employed to store the surgical data, including the video(s) captured during the surgical procedures. The data collection system 150 includes one or more storage devices 152. The data collection system 150 can be a local storage system, a cloud-based storage system, or a combination thereof. Further, the data collection system 150 can use any type of cloud-based storage architecture, for example, public cloud, private cloud, hybrid cloud, and/or the like including combinations and/or multiples thereof. In some examples, the data collection system can use a distributed storage, i.e., storage devices 152 are located at different geographic locations. The storage devices 152 can include any type of electronic data storage media used for recording machine-readable data, such as semiconductor-based, magnetic-based, optical-based storage media, and/or the like including combinations and/or multiples thereof. For example, the data storage media can include flash-based solid-state drives (SSDs), magnetic-based hard disk drives, magnetic tape, optical discs, and/or the like including combinations and/or multiples thereof.

In one or more examples, the data collection system 150 can be part of the video recording system 104, or vice-versa. In some examples, the data collection system 150, the video recording system 104, and the computing system 102, can communicate with each other via a communication network, which can be wired, wireless, or a combination thereof. The communication between the systems can include the transfer of data (e.g., video data, instrumentation data, and/or the like including combinations and/or multiples thereof), data manipulation commands (e.g., browse, copy, paste, move, delete, create, compress, and/or the like including combinations and/or multiples thereof), data manipulation results, and/or the like including combinations and/or multiples thereof. In one or more examples, the computing system 102 can manipulate the data already stored/being stored in the data collection system 150 based on outputs from the one or more machine learning models (e.g., phase detection, anatomical structure detection, surgical tool detection, and/or the like including combinations and/or multiples thereof). Alternatively, or in addition, the computing system 102 can manipulate the data already stored/being stored in the data collection system 150 based on information from the surgical instrumentation system 106.

In one or more examples, the video captured by the video recording system 104 is stored on the data collection system 150. In some examples, the computing system 102 curates parts of the video data being stored on the data collection system 150. In some examples, the computing system 102 filters the video captured by the video recording system 104 before it is stored on the data collection system 150. Alternatively, or in addition, the computing system 102 filters the video captured by the video recording system 104 after it is stored on the data collection system 150.

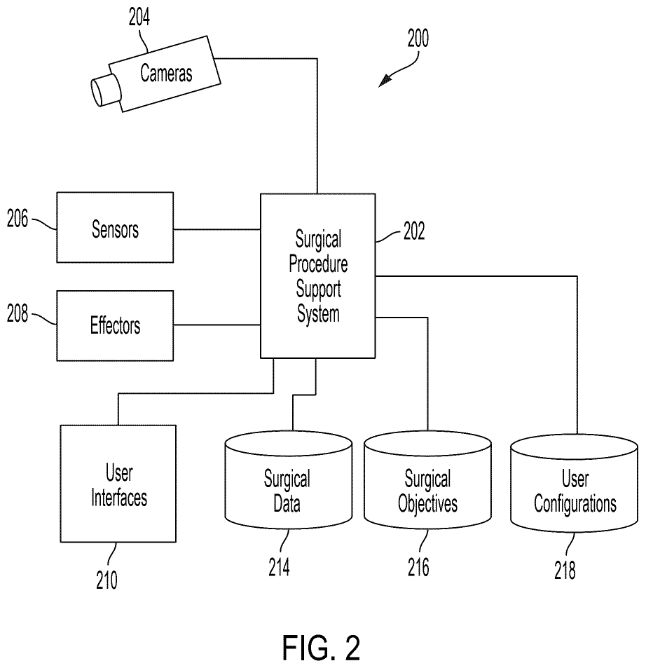

Turning now to FIG. 2, a surgical procedure system 200 is generally shown according to one or more aspects. The example of FIG. 2 depicts a surgical procedure support system 202 that can include or may be coupled to the CAS system 100 of FIG. 1. The surgical procedure support system 202 can acquire image or video data using one or more cameras 204. The surgical procedure support system 202 can also interface with one or more sensors 206 and/or one or more effectors 208. The sensors 206 may be associated with surgical support equipment and/or patient monitoring. The effectors 208 can be robotic components or other equipment controllable through the surgical procedure support system 202. The surgical procedure support system 202 can also interact with one or more user interfaces 210, such as various input and/or output devices. The surgical procedure support system 202 can store, access, and/or update surgical data 214 associated with a training dataset and/or live data as a surgical procedure is being performed on patient 110 of FIG. 1. The surgical procedure support system 202 can store, access, and/or update surgical objectives 216 to assist in training and guidance for one or more surgical procedures. User configurations 218 can track and store user preferences.

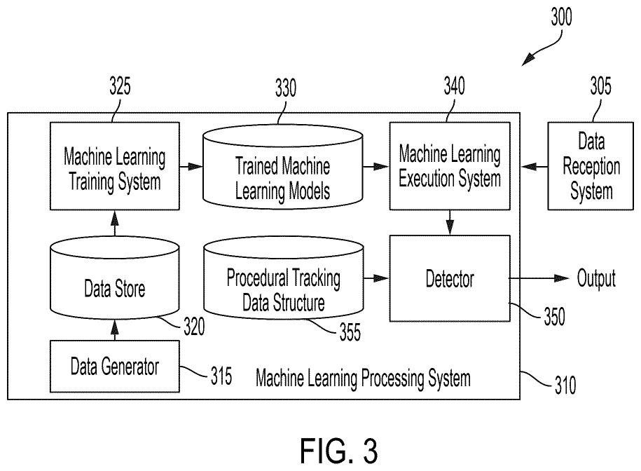

Turning now to FIG. 3, a system 300 for analyzing video and data is generally shown according to one or more aspects. In accordance with aspects, the video and data is captured from video recording system 104 of FIG. 1. The analysis can result in predicting features that include surgical phases and structures (e.g., instruments, anatomical structures, and/or the like including combinations and/or multiples thereof) in the video data using machine learning. System 300 can be the computing system 102 of FIG. 1, or a part thereof in one or more examples. System 300 uses data streams in the surgical data to identify procedural states according to some aspects.

System 300 includes a data reception system 305 that collects surgical data, including the video data and surgical instrumentation data. The data reception system 305 can include one or more devices (e.g., one or more user devices and/or servers) located within and/or associated with a surgical operating room and/or control center. The data reception system 305 can receive surgical data in real-time, i.e., as the surgical procedure is being performed. Alternatively, or in addition, the data reception system 305 can receive or access surgical data in an offline manner, for example, by accessing data that is stored in the data collection system 150 of FIG. 1.

System 300 further includes a machine learning processing system 310 that processes the surgical data using one or more machine learning models to identify one or more features, such as surgical phase, instrument, anatomical structure, and/or the like including combinations and/or multiples thereof, in the surgical data. It will be appreciated that machine learning processing system 310 can include one or more devices (e.g., one or more servers), each of which can be configured to include part or all of one or more of the depicted components of the machine learning processing system 310. In some instances, a part or all of the machine learning processing system 310 is cloud-based and/or remote from an operating room and/or physical location corresponding to a part or all of data reception system 305. It should be appreciated that several components of the machine learning processing system 310 are depicted and described herein. However, the components are just one example structure of the machine learning processing system 310, and that in other examples, the machine learning processing system 310 can be structured using a different combination of the components. Such variations in the combination of the components are encompassed by the technical solutions described herein.

The machine learning processing system 310 includes a machine learning training system 325, which can be a separate device (e.g., server) that stores its output as one or more trained machine learning models 330. The machine learning models 330 are accessible by a machine learning execution system 340. The machine learning execution system 340 can be separate from the machine learning training system 325 in some examples. In other words, in some aspects, devices that “train” the models are separate from devices that “infer,” i.e., perform real-time processing of surgical data using the trained machine learning models 330.

Machine learning processing system 310, in some examples, further includes a data generator 315 to generate simulated surgical data, such as a set of synthetic images and/or synthetic video, in combination with real image and video data from the video recording system 104, to generate trained machine learning models 330. Data generator 315 can access (read/write) a data store 320 to record data, including multiple images and/or multiple videos. The images and/or videos can include images and/or videos collected during one or more procedures (e.g., one or more surgical procedures). For example, the images and/or video may have been collected by a user device worn by the actor 112 of FIG. 1 (e.g., surgeon, surgical nurse, anesthesiologist, and/or the like including combinations and/or multiples thereof) during the surgery, a non-wearable imaging device located within an operating room, an endoscopic camera inserted inside the patient 110 of FIG. 1, and/or the like including combinations and/or multiples thereof. The data store 320 is separate from the data collection system 150 of FIG. 1 in some examples. In other examples, the data store 320 is part of the data collection system 150.

Each of the images and/or videos recorded in the data store 320 for performing training (e.g., generating the machine learning models 330) can be defined as a base image and can be associated with other data that characterizes an associated procedure and/or rendering specifications. For example, the other data can identify a type of procedure, a location of a procedure, one or more people involved in performing the procedure, surgical objectives, and/or an outcome of the procedure. Alternatively, or in addition, the other data can indicate a stage of the procedure with which the image or video corresponds, rendering specification with which the image or video corresponds and/or a type of imaging device that captured the image or video (e.g., and/or, if the device is a wearable device, a role of a particular person wearing the device, and/or the like including combinations and/or multiples thereof). Further, the other data can include image-segmentation data that identifies and/or characterizes one or more objects (e.g., tools, anatomical objects, and/or the like including combinations and/or multiples thereof) that are depicted in the image or video. The characterization can indicate the position, orientation, or pose of the object in the image. For example, the characterization can indicate a set of pixels that correspond to the object and/or a state of the object resulting from a past or current user handling. Localization can be performed using a variety of techniques for identifying objects in one or more coordinate systems.

The machine learning training system 325 uses the recorded data in the data store 320, which can include the simulated surgical data (e.g., set of synthetic images and/or synthetic video) and/or actual surgical data to generate the trained machine learning models 330. The trained machine learning models 330 can be defined based on a type of model and a set of hyperparameters (e.g., defined based on input from a client device). The trained machine learning models 330 can be configured based on a set of parameters that can be dynamically defined based on (e.g., continuous or repeated) training (i.e., learning, parameter tuning). Machine learning training system 325 can use one or more optimization algorithms to define the set of parameters to minimize or maximize one or more loss functions. The set of (learned) parameters can be stored as part of the trained machine learning models 330 using a specific data structure for a particular trained machine learning model of the trained machine learning models 330. The data structure can also include one or more non-learnable variables (e.g., hyperparameters and/or model definitions).

Machine learning execution system 340 can access the data structure(s) of the trained machine learning models 330 and accordingly configure the trained machine learning models 330 for inference (e.g., prediction, classification, and/or the like including combinations and/or multiples thereof). The trained machine learning models 330 can include, for example, a fully convolutional network adaptation, an adversarial network model, an encoder, a decoder, or other types of machine learning models. The type of the trained machine learning models 330 can be indicated in the corresponding data structures. The trained machine learning models 330 can be configured in accordance with one or more hyperparameters and the set of learned parameters.

The trained machine learning models 330, during execution, receive, as input, surgical data to be processed and subsequently generate one or more inferences according to the training. For example, the video data captured by the video recording system 104 of FIG. 1 can include data streams (e.g., an array of intensity, depth, and/or RGB values) for a single image or for each of a set of frames (e.g., including multiple images or an image with sequencing data) representing a temporal window of fixed or variable length in a video. The video data that is captured by the video recording system 104 can be received by the data reception system 305, which can include one or more devices located within an operating room where the surgical procedure is being performed. Alternatively, the data reception system 305 can include devices that are located remotely, to which the captured video data is streamed live during the performance of the surgical procedure. Alternatively, or in addition, the data reception system 305 accesses the data in an offline manner from the data collection system 150 or from any other data source (e.g., local or remote storage device).

The data reception system 305 can process the video and/or data received. The processing can include decoding when a video stream is received in an encoded format such that data for a sequence of images can be extracted and processed. The data reception system 305 can also process other types of data included in the input surgical data. For example, the surgical data can include additional data streams, such as audio data, RFID data, textual data, measurements from one or more surgical instruments/sensors, and/or the like including combinations and/or multiples thereof, that can represent stimuli/procedural states from the operating room. The data reception system 305 synchronizes the different inputs from the different devices/sensors before inputting them in the machine learning processing system 310.

The trained machine learning models 330, once trained, can analyze the input surgical data, and in one or more aspects, predict and/or characterize features (e.g., structures) included in the video data included with the surgical data. The video data can include sequential images and/or encoded video data (e.g., using digital video file/stream formats and/or codecs, such as MP4, MOV, AVI, WEBM, AVCHD, OGG, and/or the like including combinations and/or multiples thereof). The prediction and/or characterization of the features can include segmenting the video data or predicting the localization of the structures with a probabilistic heatmap. In some instances, the one or more trained machine learning models 330 include or are associated with a preprocessing or augmentation (e.g., intensity normalization, resizing, cropping, and/or the like including combinations and/or multiples thereof) that is performed prior to segmenting the video data. An output of the one or more trained machine learning models 330 can include image-segmentation or probabilistic heatmap data that indicates which (if any) of a defined set of structures are predicted within the video data, a location and/or position and/or pose of the structure(s) within the video data, and/or state of the structure(s). The location can be a set of coordinates in an image/frame in the video data. For example, the coordinates can provide a bounding box. The coordinates can provide boundaries that surround the structure(s) being predicted. The trained machine learning models 330, in one or more examples, are trained to perform higher-level predictions and tracking, such as predicting a phase of a surgical procedure and tracking one or more surgical instruments used in the surgical procedure.

While some techniques for predicting a surgical phase (“phase”) in the surgical procedure are described herein, it should be understood that any other technique for phase prediction can be used without affecting the aspects of the technical solutions described herein. In some examples, the machine learning processing system 310 includes a detector 350 that uses the trained machine learning models 330 to identify various items or states within the surgical procedure (“procedure”). The detector 350 can use a particular procedural tracking data structure 355 from a list of procedural tracking data structures. The detector 350 can select the procedural tracking data structure 355 based on the type of surgical procedure that is being performed. In one or more examples, the type of surgical procedure can be predetermined or input by actor 112. For instance, the procedural tracking data structure 355 can identify a set of potential phases that can correspond to a part of the specific type of procedure as “phase predictions”, where the detector 350 is a phase detector.

In some examples, the procedural tracking data structure 355 can be a graph that includes a set of nodes and a set of edges, with each node corresponding to a potential phase. The edges can provide directional connections between nodes that indicate (via the direction) an expected order during which the phases will be encountered throughout an iteration of the procedure. The procedural tracking data structure 355 may include one or more branching nodes that feed to multiple next nodes and/or can include one or more points of divergence and/or convergence between the nodes. In some instances, a phase indicates a procedural action (e.g., surgical action) that is being performed or has been performed and/or indicates a combination of actions that have been performed. In some instances, a phase relates to a biological state of a patient undergoing a surgical procedure. For example, the biological state can indicate a complication (e.g., blood clots, clogged arteries/veins, and/or the like including combinations and/or multiples thereof), pre-condition (e.g., lesions, polyps, and/or the like including combinations and/or multiples thereof). In some examples, the trained machine learning models 330 are trained to detect an “abnormal condition,” such as hemorrhaging, arrhythmias, blood vessel abnormality, and/or the like including combinations and/or multiples thereof.

Each node within the procedural tracking data structure 355 can identify one or more characteristics of the phase corresponding to that node. The characteristics can include visual characteristics. In some instances, the node identifies one or more tools that are typically in use or available for use (e.g., on a tool tray) during the phase. The node also identifies one or more roles of people who are typically performing a surgical task, a typical type of movement (e.g., of a hand or tool), and/or the like including combinations and/or multiples thereof. Thus, detector 350 can use the segmented data generated by machine learning execution system 340 that indicates the presence and/or characteristics of particular objects within a field of view to identify an estimated node to which the real image data corresponds. Identification of the node (i.e., phase) can further be based upon previously detected phases for a given procedural iteration and/or other detected input (e.g., verbal audio data that includes person-to-person requests or comments, explicit identifications of a current or past phase, information requests, and/or the like including combinations and/or multiples thereof).

The detector 350 can output predictions, such as a phase prediction associated with a portion of the video data that is analyzed by the machine learning processing system 310. The phase prediction is associated with the portion of the video data by identifying a start time and an end time of the portion of the video that is analyzed by the machine learning execution system 340. The phase prediction that is output can include segments of the video where each segment corresponds to and includes an identity of a surgical phase as detected by the detector 350 based on the output of the machine learning execution system 340. Further, the phase prediction, in one or more examples, can include additional data dimensions, such as, but not limited to, identities of the structures (e.g., instrument, anatomy, and/or the like including combinations and/or multiples thereof) that are identified by the machine learning execution system 340 in the portion of the video that is analyzed. The phase prediction can also include a confidence score of the prediction. Other examples can include various other types of information in the phase prediction that is output. Further, other types of outputs of the detector 350 can include state information or other information used to generate audio output, visual output, and/or commands. For instance, the output can trigger an alert, an augmented visualization, identify a predicted current condition, identify a predicted future condition, command control of equipment, and/or result in other such data/commands being transmitted to a support system component, e.g., through surgical procedure support system 202 of FIG. 2.

It should be noted that although some of the drawings depict endoscopic videos being analyzed, the technical solutions described herein can be applied to analyze video and image data captured by cameras that are not endoscopic (i.e., cameras external to the patient's body) when performing open surgeries (i.e., not laparoscopic surgeries). For example, the video and image data can be captured by cameras that are mounted on one or more personnel in the operating room (e.g., surgeon). Alternatively, or in addition, the cameras can be mounted on surgical instruments, walls, or other locations in the operating room. Alternatively, or in addition, the video can be images captured by other imaging modalities, such as ultrasound.

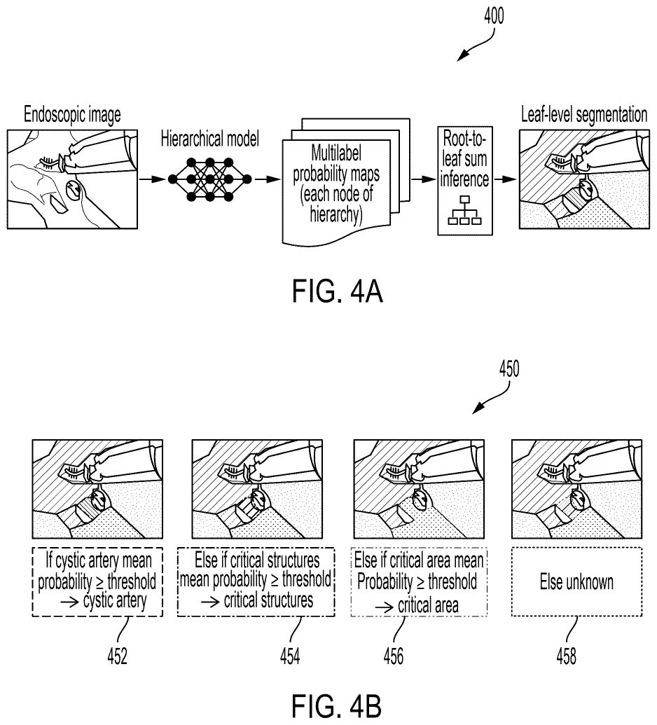

Turning now to FIG. 4A and FIG. 4B, a method for performing a hierarchical segmentation of video frames in surgical videos is depicted according to one or more aspects. As shown, FIG. 4A depicts a hierarchical model inference for a laparoscopic frame 400 and FIG. 4B depicts mixing 450 shown for a cystic artery only, where block 452 corresponds to a trained hierarchical model which processes an image to give multi-label probability maps for each node of a pre-defined hierarchy of segmentation classes. Block 454 corresponds to the higher-level critical structures class used to indicate uncertainty between cystic artery and cystic duct where a root-to-leaf sum inference is performed over each pixel to give a leaf-level segmentation map. Block 456 corresponds to the root-level critical area class that groups together the critical structures and undissected area below them, where a post-processing step is performed for each leaf-level anatomical segmentation and whereby the associated class label is updated to successively higher parent class labels in the hierarchy until sufficient prediction confidence is obtained. Block 458 corresponds to an “unknown” category used to indicate uncertainty at the root level of the hierarchy.

It should be appreciated that HSS may include arranging segmentation classes into a tree structured hierarchy for the purpose of exploiting hierarchical relationships for enhanced learning. The hierarchy, T, may be composed of nodes and edges, (V, E). Each node v∈V represents a class, while each edge (u, v)∈E represents the hierarchical relationship between two classes u, v∈V, where v is the parent mode of the child node u. In T, it may be assumed that each class node is both a parent and a child of itself, i.e., (v, v)∈E. Moreover, in T, the root nodes, V_{R}, represent the most general classes, while the leaf nodes, V_{L}, represent the most granular classes. It should also be appreciated that typical hierarchy-agnostic segmentation models map an image I∈ [symbol] to a dense feature tensor F∈ [symbol] . Subsequently, F is mapped to a dense probability tensor Y∈[0, 1]^{H×W×|V}^{_{L}}^{|} using the Softmax operator. Hierarchy-agnostic segmentation models are customarily optimized using the categorical cross-entropy (CCE) loss:

$${L}^{CCE} ( Y , T ) = - {\sum }_{i ∈ I} {\sum }_{v ∈ {𝒱}_{ℒ}} {t}_{i , v} log {y}_{i , v} , \qquad ( 1 )$$

Where T∈{0, 1}^{H×W×|V}^{_{L}}^{|} represents the ground-truth tensor. During inference, the argmax operation may be used to obtain a prediction map P∈V_{L}^{H×W}.

It is worth noting that hierarchical semantic segmentation may require a change from the multi-class classification formulation described above for hierarchy-agnostic models, to a multi-label classification formulation, i.e., rather than map each pixel to a single class from the set of leaf nodes, each pixel is now mapped to one class at each level of the hierarchy. If it is assumed that T has N levels, and each level is “complete”, i.e., contains classes that account for all possible objects in the image, then the probability tensor output may be defined by the hierarchical model as:

$$S ∈ [ 0 , 1 ] H \times W \times \vert V \vert ,$$

where S is the union of probability tensors Yi per level of the hierarchy (i.e., S=Y1∪Y2∪ . . . ∪YN). As a result, the hierarchical model is trained using a summation of CCE losses, which can be referred to as “Hiera CCE” and which may be described by:

$${L}^{HieraCCE} = {\sum }_{n ∈ N} {L}^{CCE} ( {Y}_{n} , {T}_{n} ) , \qquad ( 2 )$$

During inference, a granular prediction may be obtained, using leaf node classes, but considering the top-scoring root-to-leaf paths in the hierarchy for each pixel i, as described by:

$$\{ {v}_{1}^{*} , ... , {v}_{N}^{*} \} = {arg max}_{𝒫} {\sum }_{v ∈ 𝒫} {s}_{v} , \qquad ( 3 )$$

where, P is the set of root-to-leaf paths in the hierarchy and

$$\{ {v}_{1}^{*} , ... , {v}_{N}^{*} \} ∈ 𝒫$$

is the top scoring root-to-leaf path, with

$${v}_{N}^{*} ∈ {𝒱}_{ℒ} .$$

The leaf node class,

$${v}_{N}^{*} ,$$

may be assigned to each pixel to give a leaf-level prediction P_{L}.

It should be appreciated that while Equation (3) ensures that pixel predictions take the hierarchy into account during the inference stage, the Hiera CCE loss described by Equation (2) does not enforce the hierarchical relationships during the training stage. One approach to solving this involves applying a “tree-min” loss (“Hiera TM”) approach, where Hiera TM enforces the following two properties:
a. Positive T-Property: For each pixel, if a class is labeled positive, then all of its parent nodes in T should be labeled positive.
b. Negative T-Property: For each pixel, if a class is labeled negative, then all of its child nodes in T should be labeled negative.
   From these two properties, the following two constraints on the pixel prediction vector, s=[sᵥ]_{v∈V}∈[0, 1]^{|V|} follow:
c. Positive T-Constraint: For each pixel, if v is labeled positive and u is a parent node of v, then it should hold that sᵥ≤sᵤ.
d. Negative T-Constraint: For each pixel, if v is labeled negative, and u is a child node of v, then it should hold that 1−sᵥ≤1−sᵤ.
   These two hierarchy constraints can be incorporated in the Hiera TM loss. Therefore, given a score vector s={sᵥ}_{v∈V}∈[0, 1]^{|V|} and an associated ground-truth binary label vector t={tᵥ}_{v∈V}∈{0, 1}^{|V|} for pixel i:

$${L}^{HieraTM} = {\sum }_{v ∈ 𝒱} - {t}_{v} {log ( min ( {s}_{u} ) )}_{u ∈ {𝒜}_{v}} - ( 1 - {t}_{v} ) log {( 1 - max ( {s}_{u} ) )}_{u ∈ {𝒞}_{v}} , \qquad ( 4 )$$

where Aᵥ and Cᵥ denote parent node and child node subsets of v in T, respectively.

It should be appreciated that to better exploit the hierarchy that is imposed on class labels, a post-processing inference method (i.e., Hiera-Mix) may be performed to update the class labels in the fine-grained leaf-level prediction map to more general class labels in the parent levels of the hierarchy based on a prediction confidence threshold. For example, referring to FIG. 4B, the cystic artery segmentation may be updated to either “critical structures” (block 454), “critical area” (block 456) or “Unknown” (block 458), stopping only when the prediction confidence threshold is satisfied or exceeded. More formally, for an N-level hierarchy, T, a leaf-level prediction map, P_{L}, may be obtained by using a top scoring root-to-leaf node inference scheme and each class is iterated in P_{L}. For a class v_{N} in P_{L}, a binary mask may be defined as BN and score maps for each class in the root-to-leaf path of class v_{N} may be defined as S₁, . . . , S_{N}, with associated classes v₁, . . . , v_{N}. Thus, for each vᵢ, the class confidences, mᵢ, can be computed using the masked mean given by:

$${m}_{i} = \frac{{\sum }_{H , W} {S}_{i} ∘ {B}_{i}}{H \times W} , i = 1 , ... , N , \qquad ( 5 )$$

where H and W are the dimensions of P_{L}. Using a pre-determined confidence threshold, T, the class label v_{N} is reassigned to v_{i*}, where the index i may be determined as follows:

$${i}^{*} = max \{ i \vert {m}_{i} \geq T , i ∈ 1 , ... , N \} . \qquad ( 6 )$$

It should be appreciated that if the threshold condition is not met i.e., there is insufficient confidence at the root level, the class label is reassigned to “Unknown”.

In order to confirm this hierarchical approach, an experimental setup, according to an aspect, using 65,000 labelled frames from 1,107 separate internally collected laparoscopic cholecystectomy videos was created and evaluated. Frames were sampled from videos in windows of 30 seconds at a rate of one frames-per-second (fps) and windows were selected from across the dissection of the area containing the critical structures (hereinafter “critical area”). The following structures were labeled: critical area, cystic artery, cystic duct, liver, Rouviere's sulcus, gallbladder, and enteric structure. Additionally, the labeled frames were separated by video into training, validation, and test (80/10/10%).

In accordance with an aspect, a segmentation network with a Swin Base (Swin-B) transformer backbone (Swin Seg) was used for evaluation. Additionally, HRNet was also used compared against in ablation experiments. It should be appreciated that both networks provide common baselines for segmentation and both networks may be implemented using PyTorch 1.12. The models were optimized using the AdamW optimizer, a learning rate of 0.0001, and a “1Cycle” scheduler (such as a “OneCycleLR” in PyTorch). The models were trained for 40 epochs with a batch size of 8 and, for evaluation, the converged model at epoch 40 was used. A “balanced” sampler was used to select training examples in each epoch, where each epoch included 2,500 samples of each class label. Each of the models took approximately 24 hours to train on a 48G NVIDIA graphics processing unit (GPU) in an example. During training, the models used random image augmentations (e.g., padding, cropping, flipping, blurring, rotation, and noise). This is merely one example and many variations can be implemented according to aspects of the disclosure.

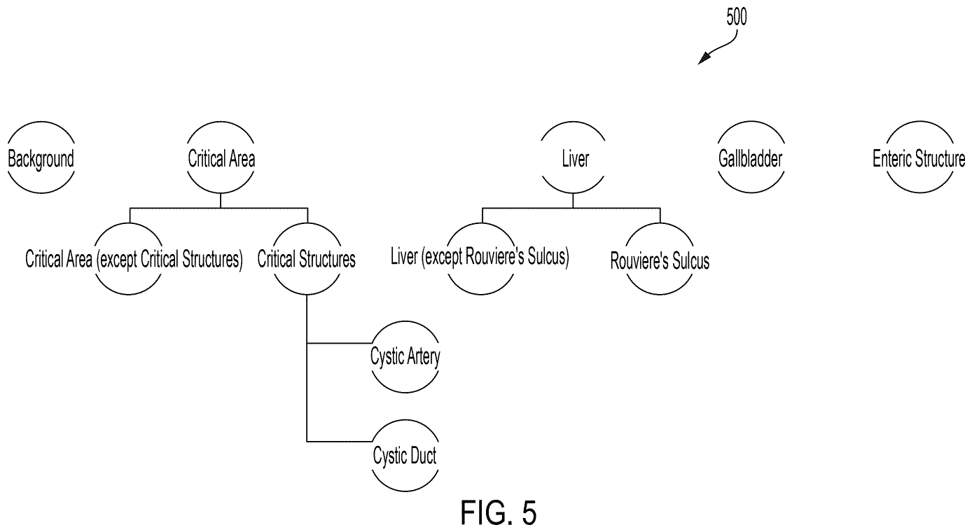

It should be appreciated that, in an aspect, for a baseline evaluation, models can be trained using the categorical cross entropy (CCE) loss. Referring to FIG. 5, a hierarchy 500 which may be used for hierarchical training and inference is shown. In an example, three hierarchical loss variants were considered during ablation experiments: Hiera CCE, Hiera TM, and the hybrid of these losses (Hiera TM+CCE), given by:

$${L}^{HieraTM + CCE} = {λ}_{1} {L}^{HieraTM} + {λ}_{2} {L}^{HieraCCE} , \qquad ( 7 )$$

where values of λ1=5 and λ2=1 were used in experiments.

In an aspect and referring to FIG. 5, the hierarchy 500 that may be used for hierarchical model training and inference is illustrated. The hierarchy groups cystic artery and cystic duct together under the critical structures, while the critical area corresponds to the undissected peritoneum-covered area that contains the critical structures, before exposure and the union of the critical structures and the undissected area below them postexposure. Through this construction, a hierarchical path from cystic artery and cystic duct to critical structures to critical area was created, thereby allowing the Hiera-Mix to trade-off between easier-to-segment but coarser labels (critical area and critical structures) and harder-to-segment but fine-grained labels (cystic artery and cystic duct).

Segmentation performance can be evaluated using a per-pixel Dice score, precision, and recall. In an example, frame-level presence detection was evaluated using per-structure F1 score, precision, and recall. In this case, for an anatomical structure to be detected as a true positive in a frame, a Dice score of 0.5 against the ground-truth annotation was required. To evaluate the proposed Hiera-Mix method, hierarchical segmentation and detection metrics were devised in which higher-level classes in the hierarchy path were allowed to count as true positives. For example, when calculating metrics for the cystic artery class, its parent classes (critical structures and critical area) are counted as true positive. The addition of a “-H” was added to denote hierarchical metrics, e.g., “Dice-H”.

Referring to Table 1 below, the impact of Hiera-Mix on the cystic artery and cystic duct using the hierarchical segmentation and detection metrics disclosed herein is shown. Through Hiera-Mix, it was observed that an increased per-pixel Dice-H and detection F1-H for both cystic artery and cystic duct occurred across both the validation and test sets. This was attributable mainly due to large increases in Precision-H, and also to small increases in Recall-H as compared to the CCE baseline (where critical area is counted as a valid true positive to allow a fair comparison). As such, Table 1 shows that Hiera-Mix improves segmentation (top) and detection (bottom) of cystic artery and cystic duct. In this aspect, the metrics assume the critical structures and critical area classes are valid predictions for cystic artery and cystic duct, where improvements are shown in green.

TABLE 1

| Anatomy | Method | Validation Set | | | Test Set | | |
|---|---|---|---|---|---|---|---|
| Pixel-Level Segmentation | | Dice-H | Precision-H | Recall-H | Dice-H | Precision-H | Recall-H |
| Cystic Artery | CCE (baseline) | 63.4% | 67.5% | 59.8% | 62.0% | 65.3% | 59.1% |
| | Hiera-Mix (ours) | 66.0% | 71.2% | 61.6% | 65.1% | 71.0% | 60.1% |
| | Difference | 2.6% | 3.7% | 1.8% | 3.1% | 5.7% | 1.0% |
| Cystic Duct | CCE (baseline) | 74.6% | 69.7% | 80.3% | 70.0% | 69.1% | 71.0% |
| | Hiera-Mix (ours) | 75.5% | 70.6% | 81.1% | 71.5% | 71.5% | 71.4% |
| | Difference | 0.9% | 0.9% | 0.8% | 1.5% | 2.4% | 0.4% |
| Image-Level Detection | | F1-H | Precision-H | Recall-H | F1-H | Precision-H | Recall-H |
| Cystic Artery | CCE (baseline) | 63.7% | 59.8% | 68.2% | 61.0% | 56.6% | 66.1% |
| | Hiera-Mix (ours) | 70.7% | 72.0% | 69.5% | 68.1% | 69.7% | 66.6% |
| | Difference | 7.0% | 12.2% | 1.3% | 7.1% | 13.1% | 0.5% |
| Cystic Duct | CCE (baseline) | 82.1% | 75.5% | 90.0% | 77.2% | 71.6% | 83.8% |
| | Hiera-Mix (ours) | 86.6% | 83.0% | 90.6% | 81.7% | 79.1% | 84.5% |
| | Difference | 4.5% | 7.5% | 0.6% | 4.5% | 7.5% | 0.7% |

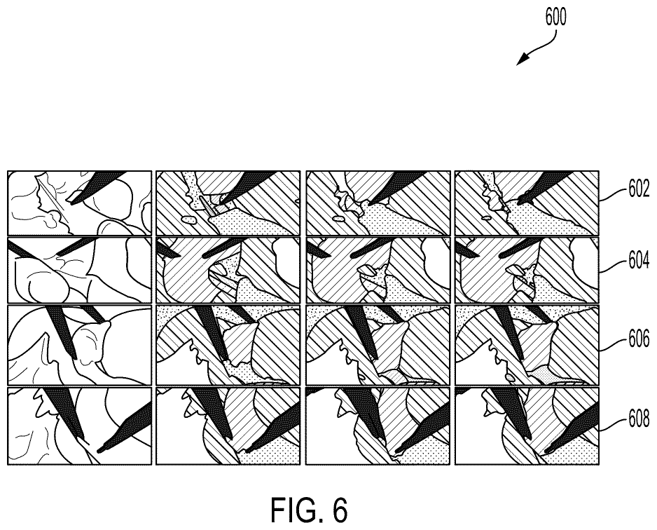

A visual comparison 600 of the CCE baseline and Hiera-Mix is shown in FIG. 6, where the top row 602 shows a frame in which the CCE model incorrectly classifies a cystic artery as cystic duct, whereas Hiera-Mix more correctly identifies it as critical structure of uncertain class. This is a difficult example since the artery is on the left of the duct in the frame, which is atypical. The middle row 604 shows a frame in which the cystic artery has been missed by the CCE-trained model, whereas the hierarchical model with Hiera-Mix has detected the cystic artery as a critical structure. The third row 606 shows a CCE-trained model detects the cystic duct, whereas the hierarchical model labels it as a critical area, as in the GT. The bottom row 608 shows a frame in which the CCE-trained model has segmented the cystic duct, prior to sufficient dissection, but the hierarchical model has labelled it gallbladder as in the ground-truth. It should be appreciated that in FIG. 6, the cystic artery may be seen as light green, the cystic duct may be seen as beige, critical structures may be seen as blue, critical areas may be seen as dark purple, the gallbladder may be seen as dark green, the liver may be seen as light brown and Rouviere's sulcus may be seen as light purple.

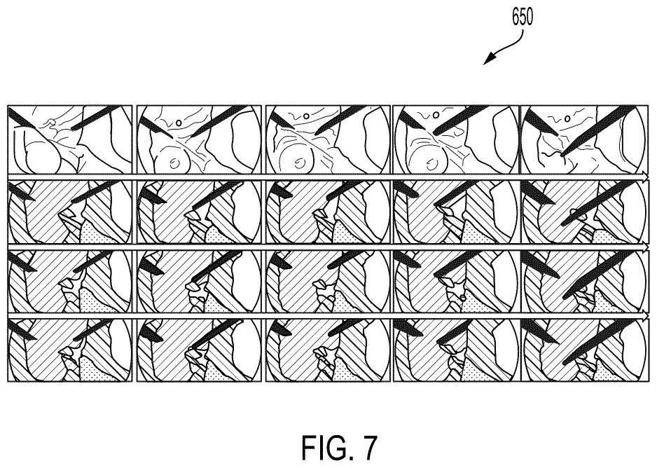

A further aspect of Hiera-Mix is shown in FIG. 7, where the CCE model 650 misses or under-segments the cystic artery in the first four frames. In comparison, Hiera-Mix uses the critical structures label to more accurately capture the cystic artery extent across the sequence in block. As can be seen, missed and under-segmentation of the cystic artery from the CCE model was observed, while Hiera-Mix better captured cystic artery extent across the sequence using cystic artery and critical structures labels. It should be appreciated that in FIG. 7, the cystic artery may be seen as light green, the cystic duct may be seen as beige, critical structures may be seen as blue, critical areas may be seen as dark purple, the gallbladder may be seen as dark green, and the liver may be seen as light brown.

Additionally, performance measured using non-hierarchical segmentation and detection metrics (only prediction of the leaf label constitutes a true positive) is shown in Table 2 below. In this case, it was observed that mixing increases the precision and reduces the recall in both segmentation (top) and detection (bottom), when examining single-class segmentation overlap for cystic artery and cystic duct due to an imposition of the confidence threshold which promotes more conservative behavior as compared to the CCE baseline. And while segmentation Dice is slightly reduced for Hiera-Mix, detection F1 is increased. Positive differences may be shown in green and negative differences may be shown in red.

TABLE 2

| Anatomy | Method | Validation Set | | | Test Set | | |
|---|---|---|---|---|---|---|---|
| Pixel-Level Segmentation | | Dice | Precision | Recall | Dice | Precision | Recall |
| Cystic Artery | CCE (baseline) | 52.5% | 61.5% | 45.9% | 54.1% | 60.8% | 48.8% |
| | Hiera-Mix (ours) | 51.2% | 63.4% | 43.0% | 53.5% | 65.0% | 45.5% |
| | Difference | 1.3% | 1.9% | 2.9% | 0.6% | 4.2% | 3.3% |
| Cystic Duct | CCE (baseline) | 65.6% | 65.3% | 65.9% | 61.7% | 64.9% | 58.7% |
| | Hiera-Mix (ours) | 65.2% | 65.7% | 64.6% | 60.5% | 66.2% | 55.8% |
| | Difference | 0.4% | 0.4% | 1.3% | 1.2% | 1.3% | 2.0% |
| Image-Level Detection | | F1 | Precision | Recall | F1 | Precision | Recall |
| Cystic Artery | CCE (baseline) | 54.4% | 53.4% | 55.4% | 53.8% | 51.6% | 56.2% |
| | Hiera-Mix (ours) | 60.8% | 74.2% | 51.4% | 57.8% | 70.3% | 49.1% |
| | Difference | 6.4% | 20.8% | 4.0% | 4.0% | 18.7% | 7.1% |
| Cystic Duct | CCE (baseline) | 74.4% | 70.0% | 79.4% | 70.8% | 67.0% | 75.0% |
| | Hiera-Mix (ours) | 78.0% | 78.7% | 77.3% | 73.8% | 76.5% | 71.2% |
| | Difference | 3.6% | 8.7% | 2.1% | 3.0% | 9.5% | 3.8% |

Referring to Table 3 below, segmentation and detection performance for all classes is shown for Swin Seg trained with CCE loss and Hiera TM+CCE loss. Importantly, for classes without hierarchical relationships, broadly similar performances were observed for the two losses, across both the validation and test sets. Ablation experiments were run initially to determine the model and hierarchical loss to use in further experiments. The mean Dice score over all classes is shown in Table 4 below. It was observed that the optimal configuration is Swin Seg trained with hybrid hierarchical loss, Hiera TM+CCE. Therefore, the results shown in Tables 1, 2, and 3, and FIGS. 4 and 5 used this approach.

TABLE 3

| | Validation Set | | | Test Set | | |
|---|---|---|---|---|---|---|
| Other Anatomy | CCE | Hiera | Diff | CCE | Hiera | Diff |
| Rouviere's Sulcus | 32.6% | 39.9% | 7.3% | 44.1% | 49.0% | 4.9% |
| Liver | 94.6% | 94.5% | 0.1% | 95.0% | 94.9% | 0.1% |
| Gallbladder | 91.1% | 90.9% | 0.2% | 91.0% | 90.9% | 0.1% |
| Enteric Structure | 79.1% | 79.5% | 0.4% | 76.6% | 76.1% | 0.5% |
| Rouviere's Sulcus | 29.9% | 34.0% | 4.1% | 40.2% | 41.6% | 1.4% |
| Liver | 98.0% | 97.9% | 0.1% | 98.6% | 98.7% | 0.1% |
| Gallbladder | 97.1% | 97.2% | 0.1% | 97.2% | 97.5% | 0.3% |
| Enteric Structure | 71.2% | 72.1% | 0.9% | 69.3% | 68.4% | 0.9% |

TABLE 4

| | | Validation |
|---|---|---|
| Model | Loss | Set |
| Swin Seg | Hiera CCE | 70.4% |
| | Hiera TM | 70.4% |
| | Hiera TM + CCE | 71.4% |
| HRNet | Hiera CCE | 70.5% |
| | Hiera TM | 69.9% |
| | Hiera TM + CCE | 70.7% |

Hierarchical segmentation with mixing (Hiera-Mix) allows the segmentation model to reflect class label uncertainty in its segmentation output, such as marking an anatomic structure as “critical structures” when it is unclear whether the structure is a cystic artery or cystic duct. Improved segmentation and detection accuracy of the cystic artery and cystic duct can result from the method, when evaluated over the sub-hierarchy for each structure. Increased precision from using Hiera-Mix implies a reduction in false-positive predictions, while increased recall suggests Hiera-Mix more often classes cystic artery and cystic duct as “critical area” at the least, compared to the model trained using the standard categorical cross-entropy (CCE) loss. The latter benefit may be due to reinforcing the belonging of cystic artery and cystic duct to the critical area during hierarchical model training.

The hierarchy for laparoscopic cholecystectomy creates a pathway from critical area to critical structures, to distinguished cystic artery and cystic duct. Hiera-Mix applied to laparoscopic cholecystectomy aims to enforce the belonging of the critical structures to the critical area, reduce premature detection of the critical structures, and reduce misidentification of the cystic artery and cystic duct. Through Hiera-Mix, increased per-pixel Dice-H and detection F1-H can be observed, attributable to large increases in precision and smaller increases in recall, compared to the CCE baseline, where critical area is also counted as a valid true positive to allow a fair comparison.

It should be appreciated that Hiera-Mix may allow the segmentation model to handle class label uncertainty in its segmentation, such as marking an anatomical structure as a critical structure when it is unclear whether it is cystic artery or cystic duct. Improved segmentation and detection for the cystic artery and cystic duct was observed from the hierarchical method, evaluated over the sub-hierarchy for each structure. In this aspect, increased precision-H from the hierarchical method may indicate a reduction in false-positives, reflecting a more conservative behavior as compared to the CCE-trained model baseline, while an increased recall-H may indicate that Hiera-Mix assigns a valid label more often than the CCE-trained model. This latter benefit is likely due to the hierarchical approach allowing the cystic artery and cystic duct to assume classes at multiple levels of the hierarchy during training.

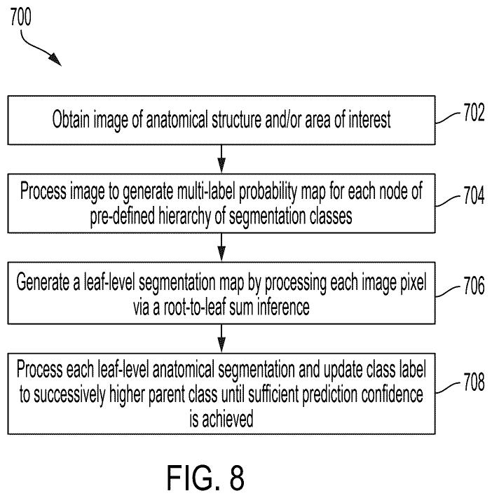

Turning now to FIG. 8, a flowchart of a method 700 for segmenting anatomy in surgical video frames using Hierarchical Semantic Segmentation (HSS) is generally shown in accordance with one or more aspects. All or a portion of method 700 can be implemented, for example, by all or a portion of CAS system 100 of FIG. 1 and/or computer system 800 of FIG. 9.

Referring to FIG. 8, according to some aspects, a method 700 for segmenting anatomy in surgical video frames using a Hierarchical Semantic Segmentation (HSS) process is shown and includes obtaining an image of an anatomical structure and/or area of interest, as shown in operational block 702. Referring again to FIG. 4A and FIG. 4B, a hierarchical model inference for a laparoscopic frame 400 is shown, where an endoscopic image is obtained for a cystic artery only. The image can be processed to generate a multi-label probability map for each node of a pre-defined hierarchy of segmentation classes, as shown in operational block 704. In this case, a trained hierarchical model can be used to process the image to give multi-label probability maps for each node of a pre-defined hierarchy of segmentation classes. A leaf-level segmentation map can be generated by performing a root-to-leaf sum inference over each of the image pixels, as shown in operational block 706. Each leaf-level anatomical segmentation can be processed, and each class label can be updated to a successively higher parent class until sufficient prediction confidence is achieved, as shown in operational block 708.

The processing shown in FIG. 8 is not intended to indicate that the operations are to be executed in any particular order or that all of the operations shown in FIG. 8 are to be included in every case. Additionally, the processing shown in FIG. 8 can include any suitable number of additional operations.

Turning now to FIG. 9, a computer system 800 is generally shown in accordance with an aspect. The computer system 800 can be an electronic computer framework comprising and/or employing any number and combination of computing devices and networks utilizing various communication technologies, as described herein. The computer system 800 can be easily scalable, extensible, and modular, with the ability to change to different services or reconfigure some features independently of others. The computer system 800 may be, for example, a server, desktop computer, laptop computer, tablet computer, or smartphone. In some examples, computer system 800 may be a cloud computing node. Computer system 800 may be described in the general context of computer-executable instructions, such as program modules, being executed by a computer system. Generally, program modules may include routines, programs, objects, components, logic, data structures, and so on that perform particular tasks or implement particular abstract data types. Computer system 800 may be practiced in distributed cloud computing environments where tasks are performed by remote processing devices that are linked through a communications network. In a distributed cloud computing environment, program modules may be located in both local and remote computer system storage media, including memory storage devices.

As shown in FIG. 9, the computer system 800 has one or more central processing units (CPU(s)) 801a, 801b, 801c, etc. (collectively or generically referred to as processor(s) 801). The processors 801 can be a single-core processor, multi-core processor, computing cluster, or any number of other configurations. The processors 801 can be any type of circuitry capable of executing instructions. The processors 801, also referred to as processing circuits, are coupled via a system bus 802 to a system memory 803 and various other components. The system memory 803 can include one or more memory devices, such as read-only memory (ROM) 804 and a random-access memory (RAM) 805. The ROM 804 is coupled to the system bus 802 and may include a basic input/output system (BIOS), which controls certain basic functions of the computer system 800. The RAM is read-write memory coupled to the system bus 802 for use by the processors 801. The system memory 803 provides temporary memory space for operations of said instructions during operation. The system memory 803 can include random access memory (RAM), read-only memory, flash memory, or any other suitable memory systems.

The computer system 800 comprises an input/output (I/O) adapter 806 and a communications adapter 807 coupled to the system bus 802. The I/O adapter 806 may be a small computer system interface (SCSI) adapter that communicates with a hard disk 808 and/or any other similar component. The I/O adapter 806 and the hard disk 808 are collectively referred to herein as a mass storage 810.

Software 811 for execution on the computer system 800 may be stored in the mass storage 810. The mass storage 810 is an example of a tangible storage medium readable by the processors 801, where the software 811 is stored as instructions for execution by the processors 801 to cause the computer system 800 to operate, such as is described hereinbelow with respect to the various Figures. Examples of computer program product and the execution of such instruction is discussed herein in more detail. The communications adapter 807 interconnects the system bus 802 with a network 812, which may be an outside network, enabling the computer system 800 to communicate with other such systems. In one aspect, a portion of the system memory 803 and the mass storage 810 collectively store an operating system, which may be any appropriate operating system to coordinate the functions of the various components shown in FIG. 9.

Additional input/output devices are shown as connected to the system bus 802 via a display adapter 815 and an interface adapter 816. In one aspect, the adapters 806, 807, 815, and 816 may be connected to one or more I/O buses that are connected to the system bus 802 via an intermediate bus bridge (not shown). A display 819 (e.g., a screen or a display monitor) is connected to the system bus 802 by a display adapter 815, which may include a graphics controller to improve the performance of graphics-intensive applications and a video controller. A keyboard, a mouse, a touchscreen, one or more buttons, a speaker, etc., can be interconnected to the system bus 802 via the interface adapter 816, which may include, for example, a Super I/O chip integrating multiple device adapters into a single integrated circuit. Suitable I/O buses for connecting peripheral devices such as hard disk controllers, network adapters, and graphics adapters typically include common protocols, such as the Peripheral Component Interconnect (PCI). Thus, as configured in FIG. 9, the computer system 800 includes processing capability in the form of the processors 801, and storage capability including the system memory 803 and the mass storage 810, input means such as the buttons, touchscreen, and output capability including the speaker 823 and the display 819.

In some aspects, the communications adapter 807 can transmit data using any suitable interface or protocol, such as the internet small computer system interface, among others. The network 812 may be a cellular network, a radio network, a wide area network (WAN), a local area network (LAN), or the Internet, among others. An external computing device may connect to the computer system 800 through the network 812. In some examples, an external computing device may be an external web server or a cloud computing node.

It is to be understood that the block diagram of FIG. 9 is not intended to indicate that the computer system 800 is to include all of the components shown in FIG. 9. Rather, the computer system 800 can include any appropriate fewer or additional components not illustrated in FIG. 9 (e.g., additional memory components, embedded controllers, modules, additional network interfaces, etc.). Further, the aspects described herein with respect to computer system 800 may be implemented with any appropriate logic, wherein the logic, as referred to herein, can include any suitable hardware (e.g., a processor, an embedded controller, or an application-specific integrated circuit, among others), software (e.g., an application, among others), firmware, or any suitable combination of hardware, software, and firmware, in various aspects. Various aspects can be combined to include two or more of the aspects described herein.

Aspects disclosed herein may be a system, a method, and/or a computer program product at any possible technical detail level of integration. The computer program product may include a computer-readable storage medium (or media) having computer-readable program instructions thereon for causing a processor to carry out various aspects.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device, such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network, and/or a wireless network. The network may comprise copper transmission cables, optical transmission fibers, wireless transmission, routers, firewalls, switches, gateway computers, and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present disclosure may be assembler instructions, instruction-set-architecture (ISA) instructions, machine instructions, machine-dependent instructions, microcode, firmware instructions, state-setting data, configuration data for integrated circuitry, or either source-code or object code written in any combination of one or more programming languages, including an object-oriented programming language, such as Smalltalk, C++, high-level languages such as Python, or the like, and procedural programming languages, such as the “C” programming language or similar programming languages. The computer-readable program instructions may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some aspects, electronic circuitry including, for example, programmable logic circuitry, field-programmable gate arrays (FPGA), or programmable logic arrays (PLA) may execute the computer-readable program instruction by utilizing state information of the computer-readable program instructions to personalize the electronic circuitry, in order to perform aspects of the present disclosure.

Aspects are described herein with reference to flowchart illustrations and/or block diagrams of methods, apparatus (systems), and computer program products according to aspects of the disclosure. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a computer system, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other devices to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various aspects. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various aspects have been presented for purposes of illustration but are not intended to be exhaustive or limited to the aspects disclosed. Many modifications and variations will be apparent to those of ordinary skill in the art without departing from the scope and spirit of the described aspects. The terminology used herein was chosen to best explain the principles of the aspects, the practical application or technical improvement over technologies found in the marketplace, or to enable others of ordinary skill in the art to understand the aspects described herein.

Various aspects are described herein with reference to the related drawings. Alternative aspects can be devised without departing from the scope of this disclosure. Various connections and positional relationships (e.g., over, below, adjacent, etc.) are set forth between elements in the following description and in the drawings. These connections and/or positional relationships, unless specified otherwise, can be direct or indirect, and the present disclosure is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains,” or “containing,” or any other variation thereof are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any aspect or design described herein as “exemplary” is not necessarily to be construed as preferred or advantageous over other aspects or designs. The terms “at least one” and “one or more” may be understood to include any integer number greater than or equal to one, i.e., one, two, three, four, etc. The terms “a plurality” may be understood to include any integer number greater than or equal to two, i.e., two, three, four, five, etc. The term “connection” may include both an indirect “connection” and a direct “connection.”

The terms “about,” “substantially,” “approximately,” and variations thereof are intended to include the degree of error associated with measurement of the particular quantity based upon the equipment available at the time of filing the application. For example, “about” can include a range of +8% or 5%, or 2% of a given value.

For the sake of brevity, conventional techniques related to making and using aspects may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

It should be understood that various aspects, and/or parts of the aspects, disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium, such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), graphics processing units (GPUs), microprocessors, application-specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

While the invention has been described with reference to aspects, it should be understood by those skilled in the art that various changes may be made and equivalents may be substituted for elements thereof without departing from the scope of the invention. Moreover, the aspects or parts of the aspects may be combined in whole or in part without departing from the scope of the invention. In addition, many modifications may be made to adapt a particular situation or material to the teachings of the invention without departing from the scope thereof. Therefore, it is intended that the invention not be limited to the particular aspects disclosed as contemplated for carrying out this invention, but that the invention will include all aspects falling within the scope of the appended claims. Moreover, unless specifically stated any use of the terms first, second, etc. do not denote any order or importance, but rather the terms first, second, etc. are used to distinguish one element from another.

What is claimed is:

1. A computer-implemented method for performing a hierarchical segmentation of video frames in surgical videos, comprising:
obtaining an image of an anatomical structure, wherein the image includes a plurality of image pixels;
generating a multi-label probability map for each node of a pre-defined hierarchy of segmentation classes;
processing the plurality of image pixels to generate a leaf-level segmentation map; and
processing each leaf-level segmentation and updating a class label for each leaf-level segmentation to a higher parent class until a prediction confidence threshold is achieved.

2. The computer-implemented method of claim 1, wherein obtaining an image includes obtaining a video stream from one or more of a camera external to a patient's body, an endoscopic camera, and a laparoscopic camera.

3. The computer-implemented method of claim 1, wherein generating a multi-label probability map includes mapping each of the plurality of image pixels to a class at each level of hierarchy.

4. The computer-implemented method of claim 1, wherein processing the plurality of image pixels to generate a leaf-level segmentation map includes performing a root-to-leaf sum inference on each of the plurality of image pixels.

5. The computer-implemented method of claim 1, wherein processing each leaf-level segmentation includes determining a prediction confidence threshold level using,
$${m}_{i} = \frac{{\sum }_{H , W} {S}_{i} ∘ {B}_{i}}{H \times W} , i = 1 , ... , N ,$$
wherein H and W are dimensions of a prediction map P_{L} and i*=max{i|mᵢ≥T, i∈1, . . . , N}.

6. The computer-implemented method of claim 1, wherein processing each leaf-level segmentation includes performing a processing inference method to update class labels in a fine-grained leaf-level prediction map to more general class labels based on the prediction confidence threshold.

7. The computer-implemented method of claim 1, wherein processing each leaf-level segmentation includes repeatedly updating each leaf-level segmentation until the prediction confidence threshold is achieved.

8. A system comprising:
a data store comprising video data associated with a surgical procedure; and
a machine learning training system for training a hierarchical model to perform hierarchical segmentation of video frames in surgical videos, the system configured to:
obtain an image of an anatomical structure from the video data, wherein the image includes a plurality of image pixels;
generate a multi-label probability map for each node of a pre-defined hierarchy of segmentation classes;
process the plurality of image pixels to generate a leaf-level segmentation map; and
process each leaf-level segmentation and update a class label for each leaf-level segmentation to a higher parent class until a pre-determined prediction confidence threshold is achieved.

9. The system of claim 8, wherein the system is configured to obtain an image using at least one of a camera external to a patient's body, an endoscopic camera, and a laparoscopic camera.

10. The system of claim 8, wherein the system is configured to generate a multi-label probability map by mapping each of the plurality of image pixels to one class at each level of hierarchy.

11. The system of claim 8, wherein the system is configured to process the plurality of image pixels by performing a root-to-leaf sum inference on each of the plurality of image pixels.

12. The system of claim 8, wherein the system is configured to process each leaf-level segmentation to determine a prediction confidence threshold level based on,
$${m}_{i} = \frac{{\sum }_{H , W} {S}_{i} ∘ {B}_{i}}{H \times W} , i = 1 , ... , N ,$$
where H and W are dimensions of a prediction map P_{L} and i*=max{i|mᵢ≥T, i∈1, . . . , N}.

13. A computer program product comprising a memory device having computer executable instructions stored thereon, which when executed by one or more processors cause the one or more processors to perform a plurality of operations for performing a hierarchical segmentation of video frames in surgical videos, the plurality of operations comprising:
obtaining an image of an anatomical structure, wherein the image includes a plurality of image pixels;
generating a multi-label probability map for two or more nodes of a pre-defined hierarchy of segmentation classes;
processing the plurality of image pixels to generate a leaf-level segmentation map; and
updating a class label for at least one leaf-level segmentation to a higher parent class until a pre-determined prediction confidence threshold is achieved.

14. The computer program product of claim 13, wherein the one or more processors are configured to obtain an image using at least one of a camera disposed external to a patient's body, an endoscopic camera, and a laparoscopic camera.

15. The computer program product of claim 13, wherein the one or more processors are configured to generate a multi-label probability map by mapping one or more of the plurality of image pixels to a class at each level of hierarchy.

16. The computer program product of claim 13, wherein the one or more processors are configured to process the plurality of image pixels by performing a root-to-leaf sum inference on one or more of the plurality of image pixels.

17. The computer program product of claim 13, wherein the one or more processors are configured to process each leaf-level segmentation to determine a prediction confidence threshold level using dimensions of a prediction map.
